(19) United States
(12) Patent Application Publication
(10) Pub. No.: US 2024/0328784 A1
(43) Pub. Date: Oct. 3, 2024

(54) TRACKING DEVICE AND METHOD OF USING THE SAME

(71) Applicant: Medtronic Navigation, Inc., Louisville, CO (US)

(72) Inventor: Mohammad Miri, Longmont, CO (US)

(21) Appl. No.: 18/608,575

(22) Filed: Mar. 18, 2024

Related U.S. Application Data
(60) Provisional application No. 63/492,610, filed on Mar. 28, 2023.

(51) Int. Cl. G01C 21/16; A61B 34/20; G01P 15/18

(52) U.S. Cl. CPC G01C 21/16; A61B 34/20; G01P 15/18; A61B 2034/2048; A61B 2034/2051; A61B 2034/2055

(57) ABSTRACT

A tracking device and method of using the same includes a sensor generating inertial signals. The device further includes a controller in communication with the sensor. The controller determines a first position based on the inertial signals relative to a registration position.

### CROSS-REFERENCE TO RELATED APPLICATION

This application claims the benefit of and priority to U.S. Provisional Patent Application No. 63/492,610 filed Mar. 28, 2023, the entire disclosure of which is incorporated by reference herein.

### FIELD

The present disclosure relates to surgical navigation, and more particularly to a system for determining the position of a device, such as a tool during a procedure.

### BACKGROUND

This section provides background information related to the present disclosure which is not necessarily prior art.

Image guided medical and surgical procedures utilize patient images obtained prior to or during a medical procedure to guide a physician performing the procedure. Recent advances in imaging technology, especially in imaging technologies that produce highly-detailed, two, three, and four dimensional images (e.g., over time), such as computed tomography (CT), magnetic resonance imaging (MRI), fluoroscopic imaging (such as with a C-arm device), positron emission tomography (PET), and ultrasound imaging (US) has increased the interest in image guided medical procedures.

Guiding a tool relative to patient position is important. Medical device companies use techniques such as optical or electromagnetic (EM) navigation to help the surgical team accurately position the tool within the patient during the procedure and create visibility for out of reach areas. While both methods come with exceptional accuracy, they may have their own limitations. For optical navigation, possible loss of line of sight for optical sensors (cameras) is an example of limitation. For EM navigation, the presence of interference, such as may be caused by metal, in the EM field that may limit the accuracy of EM navigation.

### SUMMARY

This section provides a general summary of the disclosure and is not a comprehensive disclosure of its full scope or all of its features.

The present disclosure creates an independent surgical navigation technique using a three-axis accelerometer and a gyroscope so that the position and trajectory of the tool is determined without the limitations set forth above with respect other navigation methods.

In one aspect of the disclosure, a tracking device includes a sensor generating inertial signals. The device further includes a controller in communication with the sensor. The controller determines a first position based on the inertial signals relative to a registration position.

In yet another aspect of the disclosure, a method includes determining a registration position for an object, generating accelerometer signals at a sensor coupled to the object based on a movement of the object, communicating the accelerometer signals from the sensor to a controller and determining a first position with the controller based on the accelerometer signals and the registration position.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1A is an environmental view in an operating theatre that uses a tool tracking system according to the present disclosure;

FIG. 1B is a simplified block diagrammatic representation of the interaction of the tracking system of FIG. 1A;

FIG. 2 is a block diagrammatic view of the tool relative to a controller for the present disclosure;

FIG. 3 is a diagram of a vector path according to the present disclosure;

FIG. 4 is a complex form of a tool path with various segments D1-Dn;

FIG. 5 is a realistic form of an origin and path in a coordinate system;

FIG. 6 is a flowchart of a method for performing a displacement determination;

FIG. 7 is a flowchart of a method for determining trajectory and the position of a tool at various segments; and

FIG. 8 is a flow chart of a method of determining a position based on an interaction of various tracking systems.

Corresponding reference numerals indicate corresponding parts throughout the several views of the drawings.

### DETAILED DESCRIPTION

Example embodiments will now be described more fully with reference to the accompanying drawings.

The subject disclosure is directed to an exemplary embodiment of a surgical procedure on a subject, such as a human body or patient. It is understood, however, that the system and methods described herein are merely exemplary and not intended to limit the scope of the claims included herein. In various embodiments, it is understood, that the systems and methods may be incorporated into and/or used on non-animate objects. Accordingly, the exemplary illustration of a surgical procedure herein is not intended to limit the scope of the appended claims.

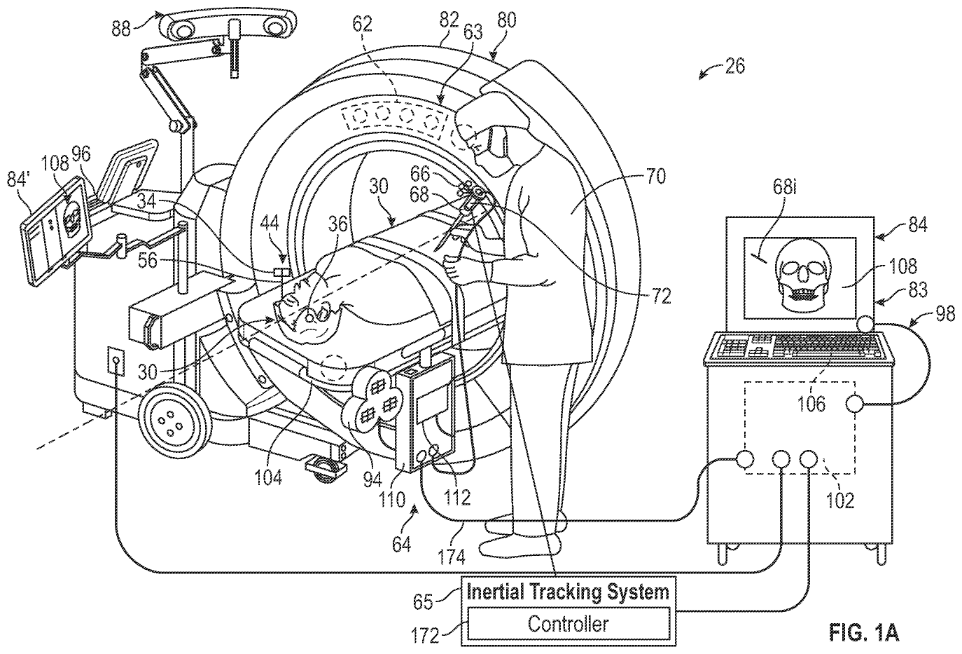

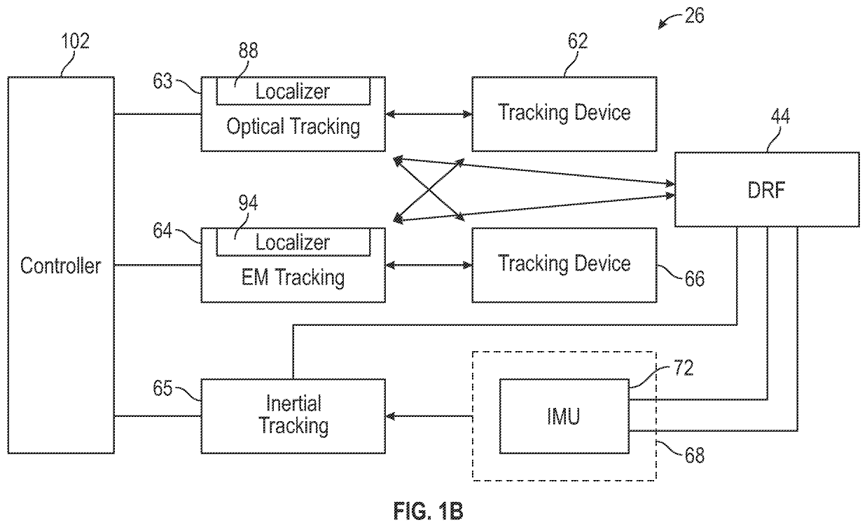

FIG. 1A is a diagrammatic view illustrating an overview of a procedure room or arena. In various embodiments, the procedure room may include a surgical suite having a navigation system 26 that can be used relative to a subject, which may be a human body or patient 30. The navigation system 26 can be used to track the location of one or more tracking devices. The tracking devices may be associated with one or more objects, such as a tool, a device, or a patient. The navigation system 26 may use various tracking modalities alone or in combination. Tracking modalities may include (1) only inertial tracking, (2) inertial tracking and only optical tracking, (3) inertial tracking and only electromagnetic tracking, or (4) inertial tracking with optical tracking and electromagnetic tracking. Details of these systems and the combinations thereof are set forth below.

Tracking devices may include those used with an optical tracking system 63, an electromagnetic tracking system 64, and an inertial tracking system 65. The optical tracking system 63 includes an optical localizer 88 and the EM tracking system 64 includes an EM array localizer 94. By way of example, a device 68 or another component may be tracked by the inertial tracking system 65 alone or in combination with the EM tracking system 64 and the optical tracking system 65. A device 68 may include a tool such as a drill, forceps, catheter, speculum or other tool operated by a user 70. The device 68 may also include an implant, such as a stent, a spinal implant or orthopedic implant. The device 68 may also include components of the system including but not limited to the imaging device 80. It should further be noted that the navigation system 26 may be used to navigate any type of device such as but not limited to an instrument, implant, stent or delivery system, including: guide wires, arthroscopic systems, orthopedic implants, spinal implants, deep brain stimulation (DBS) probes, etc. Moreover, the instruments may be used to navigate or map any region of the body. The navigation system 26 and the various instruments may be used in any appropriate procedure, such as one that is generally minimally invasive or an open procedure including cranial procedures.

An imaging device 80 may be used to acquire pre-, intra-, or post-operative or real-time image data of a subject, such as the patient 30. The image data may be used to display an image, such as an image that is reconstructed based on the image data and/or a model that is altered or reconstructed based on the image data. It will be understood, however, that any appropriate subject can be imaged and any appropriate procedure may be performed relative to the subject. In the example shown, the imaging device 80 comprises an O-arm® imaging device sold by Medtronic Navigation, Inc. having a place of business in Louisville, Colorado, USA. The imaging device 80 may have a generally annular gantry housing 82 in which an image capturing portion is moveably placed. The image capturing portion may include an x-ray source or emission portion and an x-ray receiving or image receiving portion located generally or as practically possible 180 degrees from each other and mounted on a rotor relative to a track or rail. The image capturing portion can be operable to rotate 360 degrees during image acquisition. The image capturing portion may rotate around a central point or axis, allowing image data of the patient 30 to be acquired from multiple directions or in multiple planes. The imaging device 80 can include those disclosed in U.S. Pat. Nos. 7,188,998; 7,108,421; 7,106,825; 7,001,045; and 6,940,941; all of which are incorporated herein by reference, or any appropriate portions thereof. In one example, the imaging device 80 can utilize flat plate technology having a 1,720 by 1,024 pixel viewing area.

The position of the imaging device 80, and/or portions therein such as the image capturing portion, can be precisely known relative to any other portion of the imaging device 80. The imaging device 80, according to various embodiments, can know and recall precise coordinates relative to a fixed or selected coordinate system. This can allow the imaging device 80 to know its position relative to the patient 30 or other references. In addition, as discussed herein, the precise knowledge of the position of the image capturing portion can be used in conjunction with a tracking system to determine the position of the image capturing portion and the image data relative to the tracked subject, such as the patient 30.

The imaging device 80 can also be tracked with a tracking device 62. The tracking device can be an optical, EM, and/or inertial tracking device. The image data defining an image space acquired of the patient 30 can, according to various embodiments, be inherently or automatically registered relative to an object space. The object or patient space can be the space defined by a patient 30 in the navigation system 26. The automatic registration can be achieved by including the tracking device 62 on the imaging device 80 and/or the determinable precise location of the image capturing portion. According to various embodiments, as discussed herein, imagable portions, virtual fiducial points and other features can also be used to allow for registration, automatic or otherwise. It will be understood, however, that image data can be acquired of any subject which will define the patient or subject space. Patient space is an exemplary subject space. Registration allows for a translation map and a translation between patient space and image space.

The patient 30 can also be tracked as the patient moves with a patient tracker, also referred to as a dynamic reference frame 44, that may be tracked with any appropriate tracking system, such as those disclosed herein. Alternatively, or in addition thereto, the patient 30 may be fixed within navigation space defined by the navigation system 26 to allow for registration. As discussed further herein, registration of the image space to the patient space or subject space allows for navigation of the device 68 with the image data. When navigating the device 68, a position of the device 68 can be illustrated relative to image data (e.g., superimposed on an image of the patient 30) acquired of the patient 30 on a display device 84 of the workstation 98. Various tracking systems, such as one including an optical localizer 88 or an electromagnetic (EM) localizer 94 can be used to track the device 68.

The combination of the dynamic reference frame 44 and image registration techniques compensate for movement of the subject after registration, such as anatomic distortions during movements of the anatomy associated with normal physiologic processes. The dynamic reference frame 44 can include a dynamic reference frame holder 56 and a removable tracking device 34. Alternatively, the dynamic reference frame 44 can include the tracking device 34 that can be formed integrally or separately from the DRF holder 56. Moreover, the DRF 44 can be provided as separate pieces and can be positioned at any appropriate position on the anatomy. For example, the removable tracking device 34 of the DRF 44 can be fixed to the skin of the patient 30 with an adhesive. Also, the DRF 44 can be positioned near a leg, arm, etc. of the patient 30. Thus, the DRF 44 does not need to be provided with a head frame or require any specific base or holding portion.

The tracking devices 34, 62, 66 or any tracking device as discussed herein, can include a sensor, a transmitter, or combinations thereof. Further, the tracking devices can be wired or wireless to provide a signal emitter or receiver within the navigation system. For example, the tracking device can include an electromagnetic coil to sense a field produced by the localizing array or localizer 94 or reflectors that can reflect a signal to be received by an optical localizer 88 of the optical tracking system. Nevertheless, one will understand that the tracking devices 34, 62, 66 can receive a signal, transmit a signal, or combinations thereof to provide information to the navigation system 26 to determine a position of the tracking device 34, 62, 66. The navigation system 26 can then determine a position of the tracking device, and thus the device, to allow for navigation relative to the patient and patient space. Fiducial markers 36, one of which is illustrated, may be fixed to the skin or to bone structure of the patient 30. The position of the fiducial marker 36 may be used as a reference point or registration point for the procedure. A tracked device can be used to track a position of and to allow a determination of a fiducial point on the patient 30. A processor can correlate the fiducial point on the patient to an image fiducial point in the image data. A tracking system can track at least one of the tracked devices, the dynamic reference frame, or combinations thereof.

The points that are selected to perform registration are fiducial points that are in the image that are known as image fiducial points. The image fiducial points can be produced by the fiducial marker 36 or selected landmarks, such as anatomical landmarks. The landmarks or fiducial markers 36 are identifiable in the image data and identifiable and accessible on the patient 30. The anatomical landmarks can include individual or distinct points on the patient 30 or contours (e.g., three-dimensional contours) defined by the patient 30. The fiducial markers 36 can be artificial markers that are positioned on the patient 30. The artificial landmarks, such as the fiducial markers 36, can also form part of the dynamic reference frame 44, such as those disclosed in U.S. Pat. No. 6,381,485, entitled “Registration of Human Anatomy Integrated for Electromagnetic Localization,” issued Apr. 30, 2002, herein incorporated by reference. Various fiducial marker-less systems, including those discussed herein, may not include the fiducial markers 36, or other artificial markers. The fiducial marker-less systems include a device or system to define in the physical space the landmark or fiducial points on the patient or contour on the patient. A fiducial-less and marker-less system can include those that do not include artificial or separate fiducial markers that are attached to or positioned on the patient 30.

Registration of the patient space or physical space to the image data or image space can require the correlation or matching of physical or virtual fiducial points and the image fiducial points. The physical fiducial points can be the fiducial markers 36 or landmarks (e.g., anatomical landmarks) in the substantially fiducial marker-less systems.

The registration can require the determination of the position of physical fiducial points in the patient space. The physical fiducial points can include the fiducial markers 36. The user 70 can touch the fiducial markers or devices 36 on the patient 30 or a tracking device can be associated with the fiducial markers 36 so that the tracking system 64, 65 can determine the location of the fiducial markers 36 without a separate tracked device. The physical fiducial points can also include a determined contour (e.g., a physical space 3d contour) using various techniques, as discussed herein.

The image fiducial points in the image data can also be determined. The user 70 can touch or locate the image fiducial points, either produced by imaging of the fiducial markers 36 or the anatomical landmarks. Also, various algorithms are generally known to determine the position of the image fiducial points. The image fiducial points can be produced in the image data by the fiducial markers 36, particular anatomical landmarks, or a contour (e.g., a 3D contour) of the patient 30 during acquisition of the image data.

Once the physical fiducial points and the image fiducial points have been identified, the image space and the physical space can be registered by determining a translation map therebetween. A processor, such as a processor within the workstation 98, can determine registration of the patient space to the image space. The registration can be performed according to generally known mapping or translation techniques. The registration can allow a navigated procedure using the image data.

More than one tracking system can be used to track the device 68 in the navigation system 26. According to various embodiments, these can include an electromagnetic tracking (EM) system having the EM localizer 94 and/or an optical tracking system having the optical localizer 88 and/or an inertial tracking system. Either or all of the tracking systems can be used to track selected tracking devices, as discussed herein. It will be understood, unless discussed otherwise, that a tracking device can be a portion trackable with a selected tracking system. A tracking device need not refer to the entire member or structure to which the tracking device is affixed or associated.

It is further appreciated that the imaging device 80 may be an imaging device other than the O-arm® imaging device and may include in addition or alternatively a fluoroscopic C-arm. Other exemplary imaging devices may include fluoroscopes such as bi-plane fluoroscopic systems, ceiling mounted fluoroscopic systems, cath-lab fluoroscopic systems, fixed C-arm fluoroscopic systems, isocentric C-arm fluoroscopic systems, 3D fluoroscopic systems, etc. Other appropriate imaging devices can also include MRI, CT, ultrasound, etc.

In various embodiments, an imaging device controller 96 may control the imaging device 80 and can receive the image data generated at the image capturing portion and store the images for later use. The controller 96 can also control the rotation of the image capturing portion of the imaging device 80. It will be understood that the controller 96 need not be integral with the gantry housing 82, but may be separate therefrom. For example, the controller may be a portion of the navigation system 26 that may include a processing and/or control system including a processing unit or processing system 102. The controller 96, however, may be integral with the gantry housing 82 and may include a second and separate processor, such as that in a portable computer.

The patient 30 can be fixed onto an operating table 104. According to one example, the table 104 can be an Axis Jackson® operating table sold by OSI, a subsidiary of Mizuho Ikakogyo Co., Ltd., having a place of business in Tokyo, Japan or Mizuho Orthopedic Systems, Inc. having a place of business in California, USA. Patient positioning devices can be used with the table, and include a Mayfield® clamp or those set forth in commonly assigned U.S. patent application Ser. No. 10/405,068 entitled “An Integrated Electromagnetic Navigation And Patient Positioning Device”, filed Apr. 1, 2003 which is hereby incorporated by reference.

The position of the patient 30 relative to the imaging device 80 can be determined by the navigation system 26. The tracking device 62 can be used to track and locate at least a portion of the imaging device 80, for example the gantry housing 82.

Accordingly, the position of the patient 30 relative to the imaging device 80 can be determined. Further, the location of the imaging portion can be determined relative to the housing 82 due to its precise position on the rail within the housing 82, substantially inflexible rotor, etc. The imaging device 80 can include an accuracy of within 10 microns, for example, if the imaging device 80 is an O-Arm® imaging device sold by Medtronic Navigation, Inc. having a place of business in Colorado, USA. Precise positioning of the imaging portion is further described in U.S. Pat. Nos. 7,188,998; 7,108,421; 7,106,825; 7,001,045; and 6,940,941; all of which are incorporated herein by reference,

According to various embodiments, the imaging device 80 can generate and/or emit x-rays from the x-ray source that propagate through the patient 30 and are received by the x-ray imaging receiving portion. The image capturing portion generates image data representing the intensities of the received x-rays. Typically, the image capturing portion can include an image intensifier that first converts the x-rays to visible light and a camera (e.g., a charge couple device) that converts the visible light into digital image data. The image capturing portion may also be a digital device that converts x-rays directly to digital image data for forming images, thus potentially avoiding distortion introduced by first converting to visible light.

Two-dimensional (2D) and/or three-dimensional (3D) fluoroscopic image data that may be taken by the imaging device 80 can be captured and stored in the imaging device controller 96. Multiple image data taken by the imaging device 80 may also be captured and assembled to provide a larger view or image of a whole region of a patient 30, as opposed to being directed to only a portion of a region of the patient 30. For example, multiple image data of the patient's 30 spine may be appended together to provide a full view or complete set of image data of the spine.

The image data can then be forwarded from the image device controller 96 to the navigation computer and/or processor system 102 that can be a part of a controller or workstation 98 having the display 84 and a user interface 106. It will also be understood that the image data is not necessarily first retained in the controller 96, but may also be directly transmitted to the workstation 98. The workstation 98 can provide facilities for displaying the image data as an image 108 on the display 84, saving, digitally manipulating, or printing a hard copy image of the received image data. The user interface 106, which may be a keyboard, mouse, touch pen, touch screen or other suitable device, allows the user 70 to provide inputs to control the imaging device 80, via the image device controller 96, or adjust the display settings of the display 84. The workstation 98 may also direct the image device controller 96 to adjust the image capturing portion of the imaging device 80 to obtain various two-dimensional images along different planes in order to generate representative two-dimensional and three-dimensional image data.

With continuing reference to FIG. 1A, the navigation system 26 can further include the tracking system including the inertial tracking system and/or either or both of the electromagnetic (EM) localizer 94 and/or the optical localizer 88. The tracking systems may include a controller 112 and interface portion 110. The interface portion 110 can be connected to the processor system 102, which can include a processor included within a computer. The EM tracking system may include the STEALTHSTATION® AXIEM™ Navigation System, sold by Medtronic Navigation, Inc. having a place of business in Colorado, USA; or can be the EM tracking system described in U.S. Pat. No. 7,751,865 issued Jul. 6, 2010, and entitled “METHOD AND APPARATUS FOR SURGICAL NAVIGATION”; U.S. Pat. No. 5,913,820, entitled “Position Location System,” issued Jun. 22, 1999; and U.S. Pat. No. 5,592,939, entitled “Method and System for Navigating a Catheter Probe,” issued Jan. 14, 1997; all of which are herein incorporated by reference. It will be understood that the navigation system 26 may also be or include any appropriate tracking system, including a STEALTHSTATION® TREON® or S7™ tracking systems having an optical localizer, that may be used as the optical localizer 88, and sold by Medtronic Navigation, Inc. having a place of business in Colorado, USA. Other tracking systems include an acoustic, radiation, radar, etc. The tracking systems can be used according to generally known or described techniques in the above incorporated references. Details will not be included herein except when to clarify selected operation of the subject disclosure.

Wired or physical connections can interconnect the tracking systems, imaging device 80, etc. Alternatively, various portions, such as the device 68 may employ a wireless communications channel, such as that disclosed in U.S. Pat. No. 6,474,341, entitled “Surgical Communication Power System,” issued Nov. 5, 2002, herein incorporated by reference, as opposed to being coupled directly to the processor system 102. Also, the tracking devices 62, 66 can generate a field and/or signal that is sensed by the localizer(s) 88, 94. Additionally or alternatively the inertial tracking system may have a tracking device that senses motion to allow for determination of a position of the tracking device.

Various portions of the navigation system 26, such as the device 68, and others as will be described in detail below, can be equipped with at least one, and generally multiple, of the tracking devices 66. The instrument can also include more than one type or modality of tracking device 66, such as an EM tracking device and/or an optical tracking device. The tracking device 66 may also include a sensor 72 that may be an inertial measurement sensor also referred to as an inertial monitoring or measuring unit (IMU) disposed on or within the device 68. The device 68 can include a graspable or manipulable portion at a proximal end and the tracking devices may be fixed near the manipulable portion of the device 68 or at the other end of the device 68.

Additional representative or alternative localization and tracking system is set forth in U.S. Pat. No. 5,983,126, entitled “Catheter Location System and Method,” issued Nov. 9, 1999, which is hereby incorporated by reference. The navigation system 26 may be a hybrid system that includes components from various tracking systems.

A graphical representation 68i, also referred to as an icon, can be used to illustrate the location of the tool 68 device 68 relative to the image data 108. As illustrated in FIG. 1A, an inertial tracking system 65 has a controller 172 that is used to track and determine the position of device 68. As will be described in more detail below, inertial measurements may be made with selected sensors, such as an accelerometer or an orientation sensor or a plurality thereof may be used to determine the position of the tool within the patient 30. The inertial tracking system 65 may also be used together with the optical and EM systems to determine a position of the device and/or patient. Although shown as a separate component, the controller 172 may be incorporated into and/or in communication with the controller 102 of the workstation 98.

Referring now to FIG. 1B, the navigation system 26 is illustrated in a simplified diagrammatic view. In this example, the controller is in communication with the optical tracking system 63, the electromagnetic tracking system 64, and the inertial tracking system 65. The tracking devices 62 and 66 may be tracked by the optical tracking system 63, the electromagnetic tracking system 64, and the inertial tracking system 65. The DRF 44 may also be in communication with the optical tracking system 63 and the electromagnetic tracking system 64. Likewise, the DRF 44 may also be used by the inertial tracking system 65 to locate or register the initial starting point during a procedure. The IMU 72 communicates acceleration signals and gyroscopic signals to the inertial tracking system 65, as will be described in more detail below, the localizers 88 and 94 may be used to determine the positions of the tracking devices 62 and 66. The optical tracking system 63 and the EM tracking system 64 use the DRF 44 as a reference.

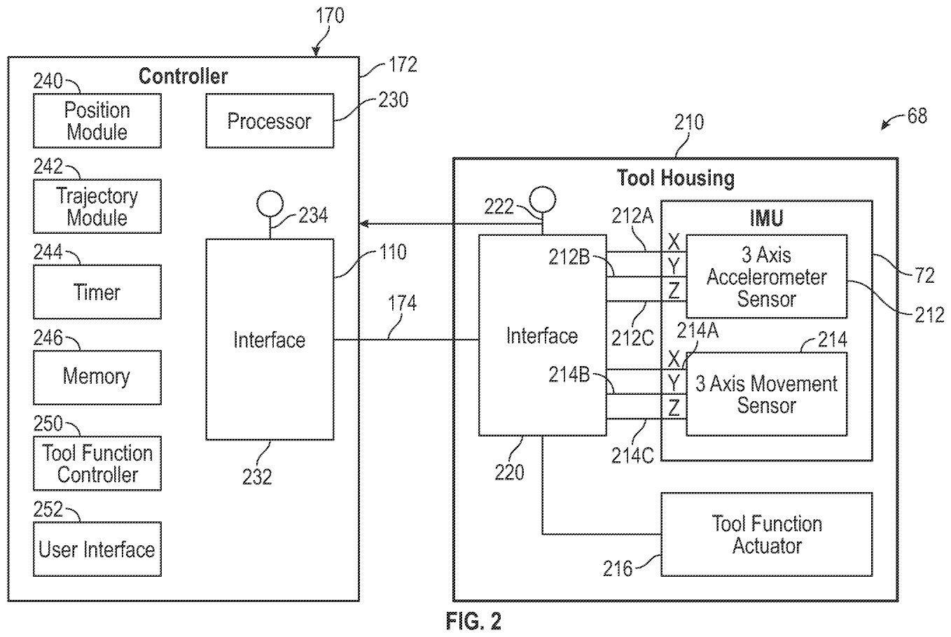

Referring now to FIG. 2, a tool housing 210 of the device 68 is illustrated in further detail. The tool housing 210 has the sensor 72 disposed therein. While the discussion herein refers to the tool housing 210 and/or a tool including the tool housing 210, the tool need not be a working instrument for a procedure. The tool may be the DRF 44, the image tracker 62, and/or any other appropriate tracking device. Thus, the sensor 72, as discussed herein, may be used to sense movement and/orientation and the information may be used to determine a path and/or trajectory of any appropriate device.

The sensor 72, as mentioned above, may be an inertial measurement unit that includes a three-axis accelerator sensor 212 that generates three measured acceleration signals corresponding to a X-axis, a Y-axis, and a Z-axis relative to the sensor and the position into which the three-axis accelerometer sensor 212 is positioned within the tool housing 210. The three axes of accelerometer sensor 212 generate accelerometer signals comprising an X-axis acceleration signal 212A, a Y-axis acceleration signal 212B and a Z-axis acceleration signal 212C. The acceleration axis signals correspond to the acceleration along respective axes.

The sensor 72 may also include a three-axis orientation sensor 214. The three-axis orientation sensor 214 generates orientation signals that correspond to the orientation about the three axes. That is, the three-axis orientation sensor 214 generates a orientation signal around the X-axis and therefore generates a X-axis orientation signal 214A, a Y-axis orientation signal 214B and a Z-axis orientation signal 214C. In various embodiments, the orientation sensor may be a gyroscope.

The tool housing 210 may also include a tool function actuator 216. The tool function actuator 216 operates the function of the specialized tool such as the motor movement of a drill or other types of functions. The sensor 72 and the tool function actuator 216 are coupled to an interface 220. The interface 220 may be a wired interface that communicates through the wire 174 or a wireless interface that communicates through an antenna 222. The interface may be part of the device tracking system 65 that includes the controller 172 as mentioned above. The sensor 72 may communicate through a wire or wirelessly.

The controller 172 of the device tracking system 65 may include a processor 230 that may be a processor module of any appropriate type and may be microprocessor-based. The processor 230 is designed and/or able to execute instructions as a program to perform a plurality of steps to determine the position or trajectory or both of the device 68. The signals from the sensor 72 are communicated from the interface 220 to the interface 232 within the controller 172. The interface 232 may be wired or wireless signal. In the case of a wireless signal, an antenna 234 receives the signals from the antenna 222. The interface 232 communicates the signals to various modules disposed within the controller 172.

A position module 240 is used to determine the location or position of the device 68 at the end of each movement segment based upon the sensor signals. Likewise, a trajectory module 242 is used to determine a trajectory of the tool. As will be described in more detail below, the position module 240 and the trajectory module 242 may be used to determine the position of the tool at various segments of the path of travel.

A timer 244 may be used to time various functions including the difference in time between an initial and end time of the different segments of the path of travel. The timer may also be used to determine or time the segments of travel, such as 1 second, 0.1 second, 0.01 second, 0.02 seconds, 0.001 second, etc. Any appropriate time segment may be measured and/or predetermined.

A memory 246 may also be included within or associated with the controller 172. The memory 246 may be used to store various values including an initial position of a tool, an end position of the tool which may become the initial position of the next segment for determination, as described below, a velocity at the end point of the last segment.

A tool function controller 250 may also be included within the controller 172. The tool function controller 250 may communicate control signals from a user interface 252 that are provided by the user 70. The user interface 252 may, for example, be a foot pedal, button, dial or switch that is used to control the function of the tool function actuator 216. By providing the position and trajectory at the position module 240 and the trajectory module 242, accurate positioning of the tool relative to the patient can be used prior to operating the tool to provide the various functions of the tool.

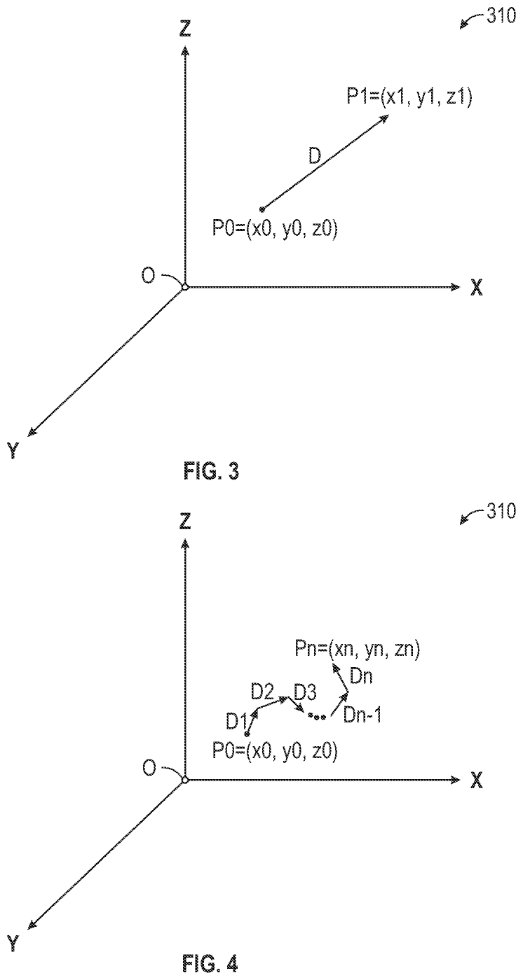

Referring now to FIG. 3, an ordinate system 310 is illustrated having an origin O and a X-axis, Y-axis, and a Z-axis. An initial position P0 and D vector are illustrated. Point P1 may be calculated by the formula:

$$P 1 = P 0 + \vec{D}$$

where P0 is known as its coordinates x0, y0 and z0 and vector D (\vec{D}) is known as Δx, Δy and Δz. Therefore, P1 will be known as x1=x0+Δx, y1=y0+Δy and z1=z0+Δz. The acceleration in each of the 3 ordinate direction is used to determine the first end location (X, Y, Z). The velocity or trajectory at the end of the segment may also be determined. As illustrated in FIG. 3, movement to the end point P1 may be a single straight line movement along vector D.

Referring now to FIG. 4, a more complex form of motion is illustrated. As illustrated in FIG. 4, movement to end point Pn may be a plurality of straight line vectors that are not along a single straight vector. In FIG. 4, to know the end destination a displacement vector at each time D1-Dn is determined. If the motion is discrete as shown in FIG. 4, the tracking of the final location is as described here. However, in reality the motion may be a continuous path on a curve.

$$Pn = P 0 + {\sum }_{1}^{n} \vec{Dn}$$

The D vectors are the displacement at each Δt (time). If the Δt shrinks toward zero (dt), the discrete path goes toward a curved realistic shape. In this case, the D vector can be described as δD. In a practical sense, the size of each segment must be large enough to enable the processor to make the acceleration calculation and allow the communication of data to be completed before the next communication is performed. Therefore, the size of the segment depends on the clock speed of the processor and the frequency of the communication.

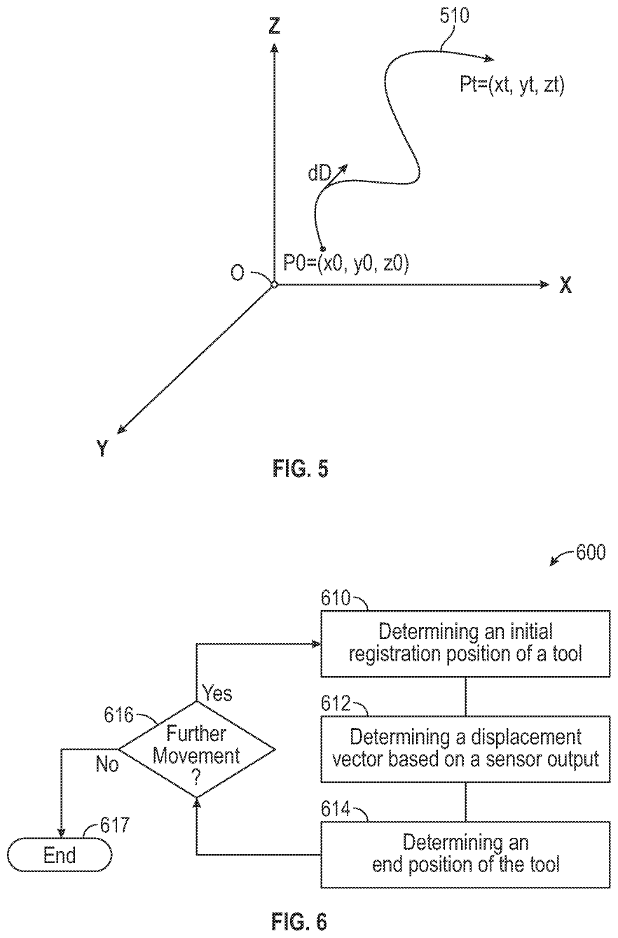

Referring now to FIG. 5, a curved path from an initial registration point to a destination is set forth. The realistic path may be one that occurs due to free hand movement of the tool by the user 70. It is understood, however, that various systems, such as a robotic system may have a realistic movement that mimics the movement illustrated in FIG. 4. To find the destination, according to various embodiments, at least one of two methods may be used. The first is to find out the result vector and use it like a one-step movement. The second is to calculate the target point for each step. The advantage of the first method is there are fewer calculations. The advantage of the second method is to provide a real time tracking of the target point.

The arithmetic representation is

$$\vec{D} = {\int }_{0}^{t} \vec{D} ( t ) dt \\ {P}_{t} = \vec{D} + {P}_{0}$$

Herein the movement is a vector D that is the integral (i.e., summation of movement) during a time to time “t” from an initial time “0” as defined by the movement D as a function of time “t” at infinitely small time intervals dt. Pt is the position at the time t and equal to the vector D plus the initial positions P₀.

In the second method the arithmetic representation P_{t+dt}=\vec{D(t)}+Pₜ

According to various embodiments, such as an EM tracking system or an optical tracking system, an instantaneous position (e.g., location and orientation) relative to an origin (e.g., DRF 44) may be known based on tracking the tracking device. With the sensor 72, however, an instantaneous position may not be known. Nevertheless, the displacement over time can be calculated with the following formula:

$$\vec{d ( t )} = \frac{1}{2} \vec{a} {t}^{2} + \vec{{v}_{0}} t + \vec{{d}_{0}}$$

where \vec{d(t)} is the displacement vector after the t time, d is the acceleration vector, t is the time, \vec{v₀} is the initial speed vector and \vec{d₀} is the initial displacement vector. Assuming the only known parameters in this equation are \vec{a} and t, the \vec{d(t)} can be calculated. That is, the vector and therefore the position at the end of each segment may be determined from the segment velocity derived from the acceleration and time period of each segment. The trajectory may be determined using the same and the orientation signals.

Firstly, the goal is to determine the path from P0 to the target point Pt by finding the displacement vector at each orientation. By starting from the known P0 (registration point, e.g., at the DRF 44), the \vec{d₀} for the first \vec{d(t)} calculation is known. The \vec{d₀} for the next \vec{d(t)} calculation will be the \vec{d(t)} of the first calculation and this chain continues toward the target point.

Secondly, the motion starts from P0 (registration point) which means there is zero speed at P0. Consequently the \vec{v₀} for the first \vec{d(t)} calculation is 0 and the \vec{v₀} for the next \vec{d(t)} can be calculated according to the following formula:

\vec{v₀}=\vec{a}t

These calculations are performed for the different segments along the path toward the target point. The acceleration in this formula is a vector which includes the path of movement. The term trajectory is used to find the orientation of the device, not the path. Therefore, the orientation system, e.g., gyroscope (IMU), is used to determine the trajectory independent from the acceleration from the acceleration sensor signal.

Referring now to FIG. 6, based on the above calculations, an accelerometer alone may be enough to find the location of the end of each segment (defining the path). As discussed above, the segment may be a time segment. A typical 3-axis (at least) accelerometer such as ICM-20948 sold by TDK Corporation may provide X, Y, and Z data signal which means the acceleration vector that includes the orientation and gyroscope signals to determine the trajectory at each time. A simplified flow chart of a method 600 of determining the end position of the tool is set forth. In block 610, the initial registration position of a tool is determined. The initial registration position of the tool may be a predetermined position in the area of the operating equipment. The various types of registration systems may use this point as a known coordinate to base various movements and positions during the procedure. For example, device may be registered at a fixed fiducial point, a DRF, or a known location on a patient. The device with the sensor 72 may touch the fixed location to be registered into the coordinate system of the navigation system. The sensors 72 may be positioned as a known or predetermined pose relative to a “touch” point of the device. For example, a distal tip of the tool may be used to touch the registration or intimately point and the pose of the distal tip relative to the sensor 72 may be determined, known, and/or recalled.

In block 612, a displacement vector based upon the accelerator sensor output is determined. Following the formulas above, the acceleration and time are used to determine the end position or location of a segment. In segments other than the initial segment, the trajectory and/or velocity may also be used in the position or location determination. Based on the acceleration output, an end position of the device is determined in block 614. The end position of the device is determined at all and/or various segments of movement based upon the velocity, time period of the segment, and the acceleration in the X, Y and Z directions noting that the velocity also has X, Y, and Z components. The various segments of movement may have a time period large enough to allow the calculations to be determined and communicated in a wired or wireless fashion. The segments may be selected and/or used to ensure an appropriate position accuracy, such as millimeter or sub-millimeter accuracy. The communication speed between the two interfaces 220, 232 may be a factor in the size of the segments. When the speed of the communication may be faster and the time segments may be smaller. In block 616, a determination of whether motion has continued and, if so, the process 600 may repeat such that the determination of other positions of the device or object during the movement may be repeated. That is, blocks 610, 612 and 614 may be repeated during the procedure. If no further movement occurs in block 616, the process 600 may end in block 617. According various embodiments, a gyroscope is not required. To improve accuracy and/or determine an instantaneous orientation of the device 68, a gyroscope may be used when considering the difference between trajectory at each point and trajectory at each movement. The gyroscope may be used to assist in determining trajectory but may only be needed for determining the trajectory of the device once stopped and/or at an instant point. The orientation sensor, therefore, may not be needed to determine the path trajectory.

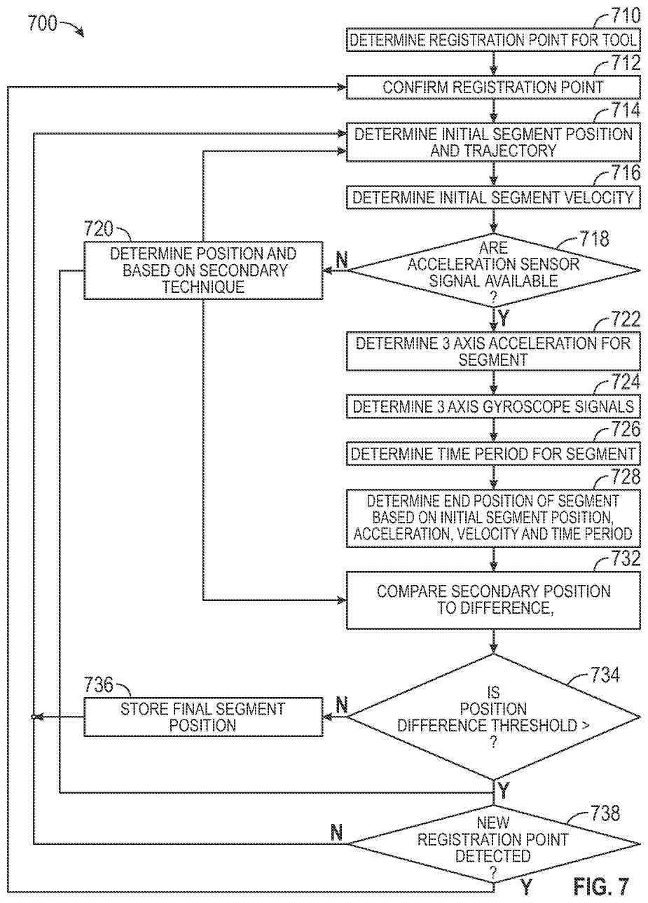

Referring now to FIG. 7, as mentioned above the accelerometer can provide the trajectory for each segment of movement of the device. However, it will not provide an instantaneous trajectory of the device at each point. In other words, the trajectory of orientation of the tool may not be known by the accelerometer signal from the sensor 212 alone. Finding the target point comes from the starting position and displacement vectors (with trajectory at each movement). However, when the target point has been reached, it is still important to identify the trajectory of the device at the target point. The target point may be a desired position for locating a tool or device for a procedure and the trajectory thereat may be desired. An example of this could be having a drill at the target point on a bone structure and being ready to start drilling. However, the direction of the drilling needs to be identified by the navigation system while the tool remains at the same/single point.

In FIG. 7, the trajectory and the X, Y, and Z position of the tool at various segments are determined. In block 710, the tool is brought into contact with a registration point. As mentioned above, a registration point may be position known by the other components of the procedure equipment. For example a dimple, recess, fiducials etc. in the DRF 44. In block 710, the registration for the tool is communicated to the controller and, more specifically, to the position module 240. The position module 240 of FIG. 2 may determine the various positions of the starting and ending of each of the segments. In block 714, an initial segment position and trajectory is determined. The initial segment position, when underway, is the registration point. However, in subsequent segments, the initial segment position is the ending segment position of the previous position. Likewise, the ending trajectory of each segment is the initial trajectory for the subsequent segment. In block 716, the initial segment velocity is determined. At the registration point, the initial velocity is 0. However, the velocity may be determined based upon the acceleration and the time. The initial velocity of the segment may be a velocity relative to all three of the axes, such as the X-axis, the Y-axis, and the Z-axis.

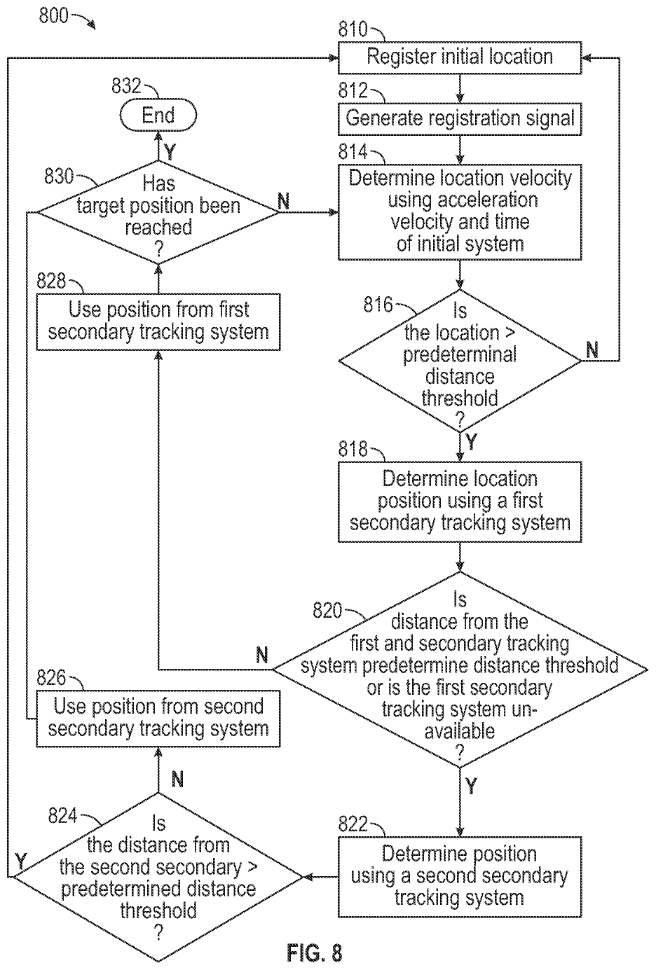

In block 718, it is determined whether the acceleration sensor signals are available. When the acceleration signals are not available, a secondary technique for determining the position and trajectory may be performed. Optical and electromagnetic determinations of the position and trajectory may be performed in a conventional manner. The interface of secondary systems is described in further detail in FIG. 8 below. Thus, the position of the tool may be determined with more than one tracking system, if selected.

In block 718, when the acceleration sensor signal is available, the three axis accelerations for each segment are determined in block 722. The three axis acceleration signals for each segment are provided by the three axes acceleration sensor signals of the three axis acceleration sensor 212.

In block 724, the orientation sensor 214 generates the gyroscope signals. The gyroscope signals are the three axis orientation signals 214A, 214B, and 214C described above.

In block 726, the time period for the present segment is determined using the timer 244 between an initial point of the segment and an end point for the segment.

In block 728, the end position of the segment based upon the initial segment position is determined as mentioned above. The end position is relative to the initial position, which is at the beginning of the current segment and/or relative to the registration position. The end position is determined based on the three axis acceleration, the velocity for the segment, and the time period of the segment.

The position of the tool may be displayed on the display. As mentioned above, the acceleration and time may be used to obtain the position. As well, the velocity at the start of the segment and time may be factored into the determination as set forth above. The orientation signals may be used to determine the trajectory of tool. The orientation sensor may be used to determine the orientation of the device alone regardless of the path.

In block 734, if the position is greater than a movement threshold, the final segment position are stored in the memory 246 in block 736. The threshold may be any appropriate threshold and may include a distance and/or orientation change value. The thresholds may be based on a realistic and/or expected maximum amount of movement in a single segment. If the position difference is greater than a movement threshold, this indicates the position difference is out of a range of a realistic or at least expected movement. For example, it is only physically possible or expected to move the tool a predetermined distance over a selected period of time. When the position are greater than a selected threshold, the position value is not used. Thereafter, block 720 uses a secondary position determination technique to determine the position of the device. After block 734, if a new registration point is detected in block 738, block 712 is repeated.

It should be noted that the determined secondary position is compared to a position difference and trajectory in block 732. That is, the system may be used in parallel with non-inertial tracking systems, such as optical or electromagnetic tracking systems, to make determinations as described relative to block 720. Based on a comparison with other techniques, when the current technique is out of a range, block 734 is performed.

Referring now to FIG. 8, a method 800 of using multiple tracking systems for locations is set forth. In block 810 the initial location or position is registered. This may be referred to as a registration point. The determination of an initial location of an object, such as a device, may be performed by one of the various methods described above. That is, various locations may be located with the device, such as by touching, include interacting with the dynamic reference frame 44 or physical locations on the body, including but not limited to the body parts themselves or fiducials mounted to the body parts may be used. Once the device or object is located at a known initial location, a registration signal may be generated in block 812. The registration signal may be confirmed to the user such as with an audible signal at the speaker 83 or a visual signal at the display 84 or both generated at the workstation 98. In this manner, the user knows that the initial registration point has been obtained and that the procedure may begin.

In block 814, the device is moved and the position after movement is determined using acceleration and trajectory. From the initial registration point, the velocity is zero (0) and the acceleration may be used alone to determine a vector in the X, Y, and Z coordinate system. The end of the vector and the magnitude of the vector corresponds to a velocity vector at the end point of the segment. As mentioned above, the path from the registration point to the target may be broken down into a plurality of segments. At the end of each segment, the location or position is determined using the change in the X, Y, and Z coordinates and the velocity. That is, a new vector for each segment is determined in the X, Y, and Z direction to obtain the new position and velocity. However, the initial velocity from the registration point is zero.

In block 816, the position (which may include a location and an orientation) of the beginning of the next segment is the end location for the previous segment. However, in order to be used as a valid position for the next segment, the end of the previous segment may be selected to be within a certain range of distance during the segment time period. The certain range may be a threshold and may generally be related to an expected or likely maximum amount of movement during a segment. The distance to the new location (or the end of the present segment) from the end of the previous segment is compared to a predetermined distance threshold in block 816. The predetermined distance threshold may be referred to as a movement threshold. The predetermined distance is a distance that corresponds to a distance greater than that which a device may be expected to physically move within a predetermined amount of time of the segment (the segment time period). For example, in one millisecond of time, may be selected that it is not possible for a device to move more than about two feet, three feet, or five feet or any appropriate amount of movement. In block 816 when the distance is not greater than a predetermined amount of time, the distance is a selected valid distance within the range of the selected threshold possibility. The system continues again in block 814 for the next segment. The next segment uses the previous X, Y, and Z locations and the velocity or the end segment locations in determining the trajectory.

In block 816 when the distance is greater than a predetermined distance, the position of the end of the segment may be determined from a first secondary tracking system in block 818. The first secondary tracking system may be an optical tracking system or an electromagnetic tracking system used to determine the position. In block 820 the position using the first secondary tracking system is compared to the predetermined distance. In a similar manner to that set forth in block 816, the position from the first secondary tracking system is compared to the predetermined distance. Again, the predetermined distance corresponds to a distance that it is not physically possible to move in the amount of time since the last segment. Block 820 may also determine if the first secondary tracking system is available.

When the first secondary tracking system is not available or the distance moved is greater than a predetermined distance, a second secondary tracking system may be used to determine the position in block 822. Block 824 determines whether the position from the second secondary tracking system is greater than the predetermined distance. When the position is not greater than the predetermined distance, block 826 uses the position from the second secondary tracking system. In block 824 when the position from the secondary tracking system is greater than the predetermined distance, the system may need reregistration by continuing with block 810.

In blocks 820 and 818, the first and second secondary tracking system may be optical or electromagnetic. When the field of view is blocked, the optical tracking system may not be available. Likewise, when the metal is in the vicinity of the object or device, electromagnetic tracking may not be available. Nevertheless, one or both of the first or secondary tracking systems, if available, may be used to confirm and/or determine a position of the device if the inertial tracking system determines a movement greater than a threshold.

In block 820 when the first position or distance is not greater than a predetermined distance (and at least one of the secondary systems are available), block 828 uses the position from the first secondary tracking system as the coordinate and the trajectory from the secondary tracking system is determined using the velocity at the predetermined positions.

The blocks are repeated until the target position has been obtained in block 830. Block 830 is performed after blocks 826 and 828. In block 830 when the target position has been obtained block 832 ends the process. When the target is reached a procedure may be performed.

However, as the system continues to move and the system has not reached the target position in block 830, the blocks 814 through 828 may be continuously performed.

In this manner, the secondary tracking systems may be used to back up the inertial tracking system when unexpected or results are obtained that are beyond those that are physically possible.

The foregoing motion detect navigation has the advantage of navigating in places with no line of sight (needed for optical) or places with or without metal objects (criteria for electromagnetic navigation). The motion detect navigation works based on an initial starting point and the path. However, if for any reason the tracking of the path is lost, the whole navigation may be lost. This can be prevented by controls on the calculations and assures there are no unreasonable steps from one point to another. If there is a large step that shows the device has moved an unreasonable amount from one point to another, the control software can void the navigation step and use another type of navigation as a back-up position determining method to recalibrate the path and define new starting point wherever available.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit that may also be referred to as a processor. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors or processor modules, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processor module” as used herein may refer to any of the foregoing structure or any other physical structure suitable for implementation of the described techniques. Also, the techniques could be fully implemented in one or more circuits or logic elements.

What is claimed is:

1. A tracking system comprising:
a sensor generating inertial signals; and
a controller in communication with the sensor, said controller determining a first position based on the inertial signals relative to a registration position.

2. The tracking system of claim 1, wherein sensor is coupled to an object;
wherein the object is configured to move from a first position to a second position.

3. The tracking system of claim 1, wherein the inertial signals include accelerometer signals and gyroscope signals and the controller determines a trajectory based on at least one of the accelerometer signals or the gyroscope signals.

4. The tracking system of claim 3, wherein the gyroscope signals comprise orientation signals about an X-axis, a Y-axis, and a Z-axis.

5. The tracking system of claim 1, wherein the accelerometer signals comprise an X-axis acceleration signal, a Y-axis acceleration signal, and a Z-axis acceleration signal.

6. The tracking system of claim 1, wherein the sensor communicates with the controller via at least one of a wire or wirelessly.

7. The tracking system of claim 1, wherein the controller determines a plurality of positions, each of the plurality of positions based on a previous position except the registration position.

8. The tracking system of claim 7, wherein the controller compares the first position to a distance threshold, when one of the first position is greater than the distance threshold, using a secondary position from a secondary tracking system.

9. The tracking system of claim 8, wherein the secondary tracking system comprises an electromagnetic tracking system.

10. The tracking system of claim 8, wherein the secondary tracking system comprises an optical tracking system.

11. The tracking system of claim 1, wherein the controller comprises a processor;
wherein the processor is configured to execute instructions to determine a pose of the sensor after a segment based on at least the inertial signals relative to an initial pose of the sensor.

12. A method comprising:
determining a registration position for an object;
generating accelerometer signals at a sensor coupled to the object based on a movement of the object;
communicating the accelerometer signals from the sensor to a controller; and
determining a first position with the controller based on the accelerometer signals and the registration position.

13. The method of claim 12, further comprising:
generating gyroscope signals at the sensor and communicating the gyroscope signal to the controller; and
determining a trajectory of the sensor based on the gyroscope signal.

14. The method of claim 13, wherein generating gyroscope signals comprises generating the gyroscope signals comprising orientation signals about an X-axis, a Y-axis, and a Z-axis.

15. The method of claim 12, wherein generating the accelerometer signals comprises generating an X-axis acceleration signal, a Y-axis acceleration signal, and a Z-axis acceleration signal.

16. The method of claim 12, further comprising determining a plurality of positions of the object after the registration position, each position of the plurality of positions based on a previous position based on the accelerometer signals and a trajectory.

17. The method of claim 16, further comprising generating a secondary position signal using a secondary tracking system.

18. The method of claim 17, further comprising comparing the first position to a distance threshold, when the first position is greater than the distance threshold, determining the position based on the secondary position signal.

19. The method of claim 18, wherein generating the secondary position signal comprises generating the secondary position signal using at least one of an electromagnetic tracking system or an optical tracking system.

20. The method of claim 14, further comprising:
determining a trajectory at the first position based at least on the generated gyroscope signals.
